(19) United States
(12) Patent Application Publication
(10) Pub. No.: US 2015/0299691 A1
(43) Pub. Date: Oct. 22, 2015

(54) STRUCTURES FOR IMMOBILISATION AND PROTECTION OF ORGANIC MOLECULES

(71) Applicant: THE UNIVERSITY OF SYDNEY, New South Wales (AU)

(72) Inventors: Aleksey Kondyurin, Penrith, New South Wales (AU); Marcela Bilek, Sutherland, New South Wales (AU)

(73) Assignee: THE UNIVERSITY OF SYDNEY, New South Wales (AU)

(21) Appl. No.: 14/443,684

(22) PCT Filed: Nov. 22, 2013
(86) PCT No.: PCT/AU2013/001347

§ 371 Date: May 19, 2015

(30) Foreign Application Priority Data
Nov. 22, 2012 (AU) 2012905128

(51) Int. Cl. C12N 11/14

(52) U.S. Cl. CPC C12N 11/14

(57) ABSTRACT

The invention relates to structures for the immobilisation and protection of organic molecules, in particular, functional biological molecules. In one aspect the invention provides a structure comprising a substrate having a nanostructured spacer on a surface thereof; and an organic molecule immobilised on the surface of the substrate. Structures of the invention have a variety of potential utilities, including in protection of catalysts used in continuous flow, in medical devices and in protection of ligands for the binding of analytes in biosensors and diagnostic devices.

### FIELD OF THE INVENTION

The present invention relates to structures for the immobilisation and protection of organic molecules, in particular, functional biological molecules.

### BACKGROUND

It is often desirable to have organic molecules, and in particular functional biological molecules immobilised to a surface.

For example, the advantages of immobilising enzymes on solid substrates for biocatalysis have long been recognised. Immobilised enzymes are widely employed in, for example, the pharmaceutical, agrochemical and food and beverage processing industries. Immobilisation of the enzyme allows for a continuous flow regime to be employed, rather than batch processing. Continuous flow allows for longer-term usage of expensive enzymes, and eliminates the labour intensive start up and shut down phases associated with batch processing.

Typically, for a continuous flow regime, enzymes are covalently attached onto a scaffold structure such that the enzymes may be stably held in the flow. Covalent attachment improves the long-term stability under processing conditions, and also makes it simpler to recover and recycle enzymes from the reaction mixture. It also allows the product concentration to be controlled, such that it may be kept below the poisoning threshold, by continuous removal of product.

In use however, the attached enzyme may be exposed to microbial attack, and the attachment may be vulnerable to hydrodynamic stresses experienced during continuous flow processing, such as, for example, in a fluidised bed reactor.

Another area where the protection of immobilised organic molecules may be sought is that of diagnostic arrays or biosensors wherein, for example, a protein, antibody or other biological molecule is attached at discrete locations on a surface to allow attachment of other molecules of interest (target molecules) and wherein means is provided for detecting the attachment of the target molecules. Protection may also be similarly necessary in other applications, such as, for example, in medical devices such as implants where the exclusion of certain proteins from adsorption to a biocompatible surface is necessary.

In seeking to address issues associated with the protection of surface immobilised molecules, one approach has sought to encapsulate the molecules, such as active enzymes, within nanoporous substrates. In recent times, a range of techniques for producing different nanostructures have been discovered. For example, a range of methods for producing nanostructures such as carbon nanotubes arrays have been described.^{1,2,3}

Production of appropriate nanoporous substrates can be challenging as the pores must be engineered carefully so that they are small enough to exclude microorganisms, whilst being sufficiently large to provide efficient transportation of reactants from the reaction mixture to the immobilised molecules.

The present invention seeks to address the above mentioned issues, in particular providing structures suitable for immobilisation and protection of organic molecules.

### SUMMARY OF THE INVENTION

In one broad form the present invention provides a structure comprising a substrate having a nanostructured spacer on a surface thereof; and an organic molecule immobilised on the surface of the substrate.

In one form, the nanostructured spacer comprises a carbon nanotube array.

In another form, the carbon nanotubes of the carbon nanotube array are aligned substantially perpendicular to the surface of the substrate.

In one form, the organic molecule is covalently attached to the surface of the substrate.

In a further form, the surface of the substrate comprises a carbon containing layer.

In one form, the organic molecule is covalently attached to the carbon containing layer.

In another form, the carbon containing layer includes any one or a combination of amorphous carbon, graphitic carbon, nano graphite or glassy carbon.

In one form, the organic molecule is a functional biological molecule.

In a further form, the functional biological molecule is an enzyme.

In one form, the average distance between the carbon nanotubes of the array is less than about 500 nm.

In another form, the average distance between the carbon nanotubes of the array is between about 5 nm to about 100 nm.

In one form, the average distance between the carbon nanotubes of the array is between about 10 nm to about 50 nm.

In one form, the carbon nanotubes have an average diameter of between from about 1 nm to about 100 nm.

In another form, the carbon nanotubes have an average diameter of between from about 10 nm to about 20 nm.

In one form, the carbon nanotubes have an average length of greater than about 500 nm.

In another form, the organic molecule is immobilised on the surface of the substrate after the carbon nanotubes are formed the substrate.

In one form, the organic molecule is a catalytic molecule and the structure is for use in continuous flow processing.

In another form, the organic molecule is a functional biological molecule and the structure is for use in a diagnostic array or biosensor.

In a further broad form the present invention provides use of a structure as a support for protecting an organic molecule, the structure comprising: a substrate having a surface for receiving and immobilising the organic molecule; and a nanostructured spacer on the surface of the substrate.

In one form, the nanostructured spacer is an array of carbon nanotubes.

In a further broad form the present invention provides a method of protecting an organic molecule, the method comprising immobilising the organic molecule to a surface of a substrate, the substrate comprising a nanostructured spacer on the surface.

In one form, the nanostructured spacer is an array of carbon nanotubes.

In one form, the organic molecule is a catalytic molecule and the method is for protecting the catalytic molecule during continuous flow processing.

In one form, the catalytic molecule is an enzyme.

In a further broad form, the present invention provides a method of producing a structure, the method comprising immobilising an organic molecule on a surface of a substrate, the surface of the substrate including a nanostructured spacer formed thereon.

In one form, the method further comprises the step of forming the nanostructured spacer on the surface of the substrate.

In one form, the nanostructured spacer is an array of carbon nanotubes.

In another form, the organic molecule is catalytic molecule.

In one form, the organic molecule is an enzyme.

### BRIEF DESCRIPTION OF THE DRAWINGS

The present invention will become better understood from the following detailed description of various non-limiting embodiments thereof, described in connection with the accompanying figures, wherein:

FIG. 1 shows SEM images of a carbon nanotube array before and after incubation in enzyme solution in accordance with Example 1, including top (a) and side (b) views of anas-prepared CNT array, and top views of the array after incubation in enzyme solution at low (c) and high (d) magnification;

FIG. 2 shows Amide I and Amide II lines in the FTIR ATR spectra of catalase immobilized on the CNT array (the spectra of the silicon wafer and spectra of a control CNT sample after incubation in buffer without protein have been subtracted). The background subtracted spectrum obtained from catalase immobilized on plasma immersion ion implantation treated UHMWPE is also shown for comparison;

FIG. 3 shows XPS spectra of a CNT array (a), CNT array with immobilized HRP (b), and CNT array after HRP immobilization and washing in SDS detergent (c) (The π-plasmon edge is indicated with arrows);

FIG. 4 shows high-resolution XPS spectra: C1s peak (a); the O1s peak (b); and the N1s peak (c) of a CNT array (black), a CNT array with immobilized HRP (blue), and a CNT array after HRP immobilization and washing in SDS detergent (red);

FIG. 5 shows a graph (compiled using the XPS spectra of FIG. 4) of the elemental content of the CNT arrays from Example 1 before and after exposure to enzyme solution;

FIG. 6 shows HRSEM images of catalase protein on carbon nanotubes and substrate: (a) and (b) show the top of the carbon nanotube spacer array, (c) and (d) show the surface underneath, viewed at an edge near the cracked substrate (d) and on the flat surface after removal of the CNTs (c);

FIG. 7 shows an AFM image (a) and its associated line profile (b) of a flat polystyrene film with adsorbed catalase macromolecules to give an indication of the size of catalase molecules; and

FIG. 8 shows Raman spectra of the nanotube array of Example 1 before (a) and after (b) incubation in protein solution, after subsequent SDS washing (c). The radial breathing mode peak is shown in (d) for the CNT before (black) and after (red) incubation in HRP protein solution and then after subsequent washing with SDS detergent (blue).

FIG. 9 shows SEM images of the upper surface of a sample after 12 h in a shaker and critical point drying (a) and after drying in open air (b). No changes in the structure of surface after critical point drying were found as compared with the as-prepared sample (FIG. 1a). (c) Schematic of the enzyme protection against microbiological attack. Enzymes, are attached on the carbon film deposited onto the substrate surface and protected by a dense and long carbon nanotube forest, while bacteria cannot reach the protected enzyme molecules. (d) and (e) Low and high resolution SEM images, respectively, of B. subtilison the surface of the nanotube pattern after 12 h of incubation. Bacteria were trapped in the upper layer of the nanotube pattern.

### DETAILED DESCRIPTION

The articles “a” and “an” are used herein to refer to one or to more than one (i.e., to at least one) of the grammatical object of the article. By way of example, “an antimicrobial agent” means one antimicrobial agent or more than one antimicrobial agent.

Documents or patent applications referred to within this specification are included herein, in their entirety, by way of reference.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

In the context of this specification, the term “about” is understood to refer to a range of numbers that a person of skill in the art would consider equivalent to the recited value in the context of achieving the same function or result.

The reference to any prior art in this specification is not, and should not be taken as, an acknowledgment or any form of suggestion that that prior art forms part of the common general knowledge in Australia.

As described and exemplified herein the present inventors have developed an approach to support and protect a catalytic molecule during continuous flow processing which involves immobilizing the catalytic molecule to a surface that includes an aligned array of carbon nanotubes (CNTs) deposited thereon. By engineering the pore size of the array (by adjusting the diameter of the CNTs and/or the spacing between them), the CNT array can be used as a means to protect the catalytic molecule from damaging particles or microorganisms by acting as a physical spacer. Microorganisms/damaging particles greater than the pore size of the array are not be able to reach the catalytic molecule, whilst dissolved reactants smaller than the pore size are able to reach and react with the immobilised catalytic molecule. In particular embodiments, the catalytic molecule is an enzyme. Those skilled in the art will appreciate that any suitable catalytic molecule may be immobilised on the surface and the scope of the invention is not limited by the identity of the catalytic molecule.

Those skilled in the art will also appreciate that, while applicable to the support and immobilisation of catalytic molecules, such as enzymes, during continuous flow processing, the structures and methods of the present invention are equally applicable to any scenario in which it is desirable to protect an organic/biological molecule immobilised on a substrate. Some further example applications include use in diagnostic arrays, biosensors and/or medical devices requiring biocompatible surfaces, such as medical implants. Persons skilled in the art will be well aware of a range of techniques applicable in biosensors or diagnostic devices including micro/nanofluidic devices for detection of binding of an analyte to an immobilised biological molecule, such as use of radioactive, colourimetric or fluorescent labels, use of metallic labels that can be sensed magnetically or the use of the detection of a change inmaterial capacitance resulting from binding (see for example Bergveld, P., “A critical evaluation of direct electrical protein detection methods”, Biosensors & Bioelectronics 6 (1991) 55-72; Chen et al, “Novel capacitive sensor:Fabrication from carbon nanotube arrays and sensing property characterization”, Sensors and Actuators B 140 (2009) 396-401; and Meng et al, “Electronic chip based on self-oriented carbon nanotube microelectrode array to enhance the selectivity of indoor air pollutants capacitive detection”, Sensors and Actuators B 153 (2011) 103-109, the disclosures of which are included herein in their entirety by way of reference.

Typically, the organic molecule to be protected is a functional biological molecule. The term “biological molecule” is intended to encompass any molecule that is derived from a biological source, is a synthetically produced replicate of a molecule that exists in a biological system, is a molecule that mimics the activity of a molecule that exists in a biological system, or that otherwise exhibits biological activity. By “functional”, it is meant that the molecule is able to exhibit at least some of the activity it would normally exhibit in a biological system. The activity exhibited may vary qualitatively and/or quantitatively from that exhibited in a biological system. Exemplary biological molecules include, but are not limited to, amino acids, peptides, proteins, glycoproteins, lipoproteins, nucleotides, oligonucleotides, nucleic acids (including DNA, RNA, LNA, PNA and combinations or modifications thereof), lipids and carbohydrates, as well as active fragments thereof. The nucleic acid may be a catalytic nucleic acid such as a ribozyme or DNAzyme. Exemplary proteins include enzymes, antibodies and antigen-binding fragments.

The size, alignment and/or density of the CNTs forming the array is such that an organic molecule immobilised on the surface of the substrate is protected, for example from contact by microorganisms, such as bacteria, and from other particles that may damage the molecule, inactivate the molecule or otherwise impair an activity of the molecule. The size, alignment and/or density of the CNTs forming the array on the substrate surface must also be such that, where the organic molecule is a functional biological molecule such as a catalytic molecule or an antibody, reagents or antigens required for the activity of the molecule are able to reach the immobilised molecule minimally impeded by the CNTs.

For example, in the case of an immobilised active enzyme, the average pore size of the CNT array may be from about 10 nm to about 20 nm so as to prevent access to the enzyme by bacteria (typically 200 to 2000 nm, such as 400 to 500 nm). Although, as those skilled in the art will appreciate, the pore size of the CNT array may be adjustable (for example by manipulating the diameter of, and spacing between, the CNTs), and would depend on the identity of the organic molecule to be immobilised and the identity of the microorganism/particle to be excluded.

In addition to the diameter and/or spacing between CNTs of the array, the length of the CNTs can contribute to the protection of the organic molecule. In particular, the length of the CNTs can provide protection not only from microbial attack or contamination but also from the hydrodynamic stresses experienced during flow processing. For protection of an immobilised enzyme, the height of the CNTs of the array may have an average height, for example, of between about 1 μm to about 20 μm. Those skilled in the art will recognise that the length of the CNTs is not limited and is generally dependent on growth/deposition time. For example, the length of the CNTs may be in the nm or mm range, or greater.

It will also be appreciated that, the diameter of the CNTs, spacing between the CNTs, and/or length of the CNTs in the array may influence the activity rate of an immobilised functional biological molecule (for example the throughput of the immobilised catalytic molecule) and therefore may be adjusted accordingly.

Typically, the organic molecule is covalently attached to the surface of the substrate, between and within the carbon′nanotubes of the CNT array. To permit covalent attachment to the surface of the substrate, an active carbon containing layer is, in one aspect of the invention, formed on the substrate surface beneath the CNT array. The carbon containing layer can include any one or a combination of amorphous carbon, graphitic carbon, nano-graphite, and glassy carbon. The present inventors understand that the carbon containing layer includes highly-reactive surface-embedded radicals that allow for the formation of covalent bonds with the organic molecules to be immobilised.

A range of CNT synthesis techniques result in the formation of a suitable carbon-containing layer beneath the CNTs. These include, but are not limited to: arc deposition from carbon electrodes in helium, hydrogen or air; chemical vapour deposition (CVD) with water, oxygen, hot-filament, mw-plasma and rf-plasma assistance; and laser ablation of a graphite target. With the appropriate selection of processing parameters, all of these methods can create an active carbon layer under the CNTs, which allows for covalent attachment of the organic molecule.

For CVD, typical precursors used for CNT synthesis may include, but are not limited to, ethylene, pyrene, benzene, methane, ethane, ethylene, acetylene, xylene, ethanol, isobutane and mixtures thereof. Typical catalysts may be, but are not limited to, Ni, Fe, Co, Cu particles. It will be appreciated by those skilled in the art that CNTs may be synthesised using CVD on some substrates without a catalyst, for example, porous alumina.

After CNT synthesis, the structure includes an array of CNTs standing on the top of a carbon containing underlayer. Thereafter, to immobilise the organic molecule, the structure having the active carbon layer and CNTs thereon is typically immersed/incubated in a solution containing the organic molecule for a period sufficient to allow covalent bonding. A person skilled in the art would appreciate that the solution containing the organic molecule may also be applied to the surface by spray coating, pipette spotting, dip coating or other means.

Preferably the solution is an aqueous solution (eg. saline), that preferably includes a buffer system compatible with maintaining the biological function of the organic molecule, such as for example a phosphate or Tris buffer.

It may then be appropriate to conduct one or more washing steps also using a biologically compatible solution or liquid, for example the same aqueous buffered solution as for the incubation (but which does not include the biological molecule), to remove any non-specifically bound material from the surface, before the structure is ready to be put to its intended use.

Persons skilled in the art would appreciate that in addition to utilising the formation of an active carbon-containing layer, other approaches can be used to covalently immobilise the organic molecule on the substrate surface. Alternative approaches include pre-treatment of the substrate such as for example, approaches described in International Patent Application Nos. PCT/AU2007/000321, PCT/AU2012/000714 and PCT/AU2008/001085.

The CNTs of the array can be single walled and/or multi walled. Furthermore, it will be appreciated that the invention is not limited to the use of un-functionalised CNTs, and in some embodiments the walls of the CNTs may be functionalised by appropriate compounds etc.

One advantage of utilising virgin or un-functionalised CNTs is that they are inactive along their length and typically do not permit covalent attachment of the organic molecule. Therefore, when the substrate with synthesised CNTs is incubated/immersed in a solution containing the organic molecules (e.g. catalytic molecule/enzyme/protein/antibody), the organic molecules are covalently immobilized on the carbon containing underlayer between and within the CNTs but not on the walls of CNTs. The organic molecules are thereby effectively spaced by the CNTs from microorganisms or other damaging particles.

Furthermore, by using an aligned CNT array, the path for the reactant to the immobilised organic molecule is substantially unimpeded. A person skilled in the art will appreciate that in certain embodiments, the CNTs of the array may not be aligned.

It is readily apparent how the described structure may be useful as a protective support for the immobilization of organic molecules, and in particular functional biological molecules such as enzymes and antibodies.

The inventors propose that the surface immobilized enzymes (or other organic molecules) would remain biologically active, retaining their enzymatic activity. A person skilled in the art would appreciate that activity of the immobilised enzyme may be tested with a suitable enzyme assay. For example, assay types may include, but are not limited to, spectrophotometric, fluorometric, and chemiluminescent assays.

Those skilled in the art would also appreciate that in place of, or in addition to carbon nanotube arrays, other suitable nanostructured spacers may be provided on the substrate surface to protect the immobilised organic molecule.

A “nanostructure” is a structure having at least one region or characteristic dimension with a dimension of less than about 500 nm, and typically between 1 to 100 nm. Exemplary nanostructures include, but are not limited to, nanowires, nanorods, nanotubes, nanofibers, branched nanostructures, nanotetrapods, tripods, bipods, nanocrystals, nanodots, quantum dots, nanoparticles, and the like. A “nanostructured” article includes, but is not limited to, an article formed of one or more nanostructures, such as, for example, an array of nanostructures, an array of carbon nanotubes or an array of carbon nanofibers.

Embodiments of the invention therefore provide a structure comprising an organic molecule immobilised on a surface of a substrate, wherein the surface of the substrate includes a nanostructured spacer thereon.

Furthermore, it will be appreciated that the substrate may be metal, semiconductor, polymer, ceramic, composite or other substrate. The substrate may, for example, take the form of a block, sheet, film, foil, tube, strand, fibre, piece or particle (eg. a nano- or micro-particle such as a nano- or micro-sphere), powder, shaped article, indented, textured or moulded article or woven fabric or massed fibre pressed into a sheet (for example like paper) of metal, semiconductor, polymer, composite and/or ceramic. The substrate can be a solid mono-material, laminated product, hybrid material or alternatively a coating on any type of base material which can be non-metallic or metallic in nature, and which may include a polymer component, such as homo-polymer, co-polymer or polymer mixture. Indeed, the substrate may also form a component of a device, such as for example a component of a diagnostic kit or detection device, a tissue, cell or organ culture scaffold or support, a biosensor, an analytical plate, an assay component, a micro- or nano-device that interacts with or includes biological components (e.g. molecular motors involving actin/myosin filaments) or a medical device such as a contact lens, a stent (eg a cardiovascular or gastrointestinal stent), a pace maker, a hearing aid, a prosthesis, an artificial joint, a bone or tissue replacement material, an artificial organ, a heart valve or replacement vessel, a suture, staple, nail, screw, bolt or other device for surgical use or other implantable or biocompatible device.

The terms “metal” or “metallic” as used herein to refer to elements, alloys or mixtures which exhibit or which exhibit at least in part metallic bonding.

The term “ceramic” as it is used herein is intended to encompass materials having a crystalline or at least partially crystalline structure formed essentially from inorganic and non-metallic compounds. They are generally formed from a molten mass that solidifies on cooling or are formed and either simultaneously or subsequently matured (sintered) by heating. Clay, glass, cement and porcelain products all fall within the category of ceramics and classes of ceramics include, for example, oxides, silicates, silicides, nitrides, carbides and phosphates.

The term “polymer” as it is used herein is intended to encompass homo-polymers, copolymers, polymer containing materials, polymer mixtures or blends, such as with other polymers and/or natural and synthetic rubbers, as well as polymer matrix composites, on their own, or alternatively as an integral and surface located component of a multi-layer laminated sandwich comprising other materials e.g. polymers, metals or ceramics (including glass), or a coating (including a partial coating) on any type of substrate material. The term “polymer” encompasses thermoset and/or thermoplastic materials as well as polymers generated by plasma deposition processes.

“Composite” materials comprehended by the present invention include those that are combinations or mixtures of other materials, such as composite metallic/ceramic materials (referred to as “cermets”) and composites of polymeric material including some metallic or ceramic content, components or elements. Such composites may comprise intimate mixtures of materials of different type or may comprise ordered, arrays or layers or defined elements of different materials.

The present disclosure is further described by reference to the following non-limiting examples.

### EXAMPLES

### Example 1

### Immobilisation of Horseradish Peroxidase and Catalase on CNT Array Covered Substrates

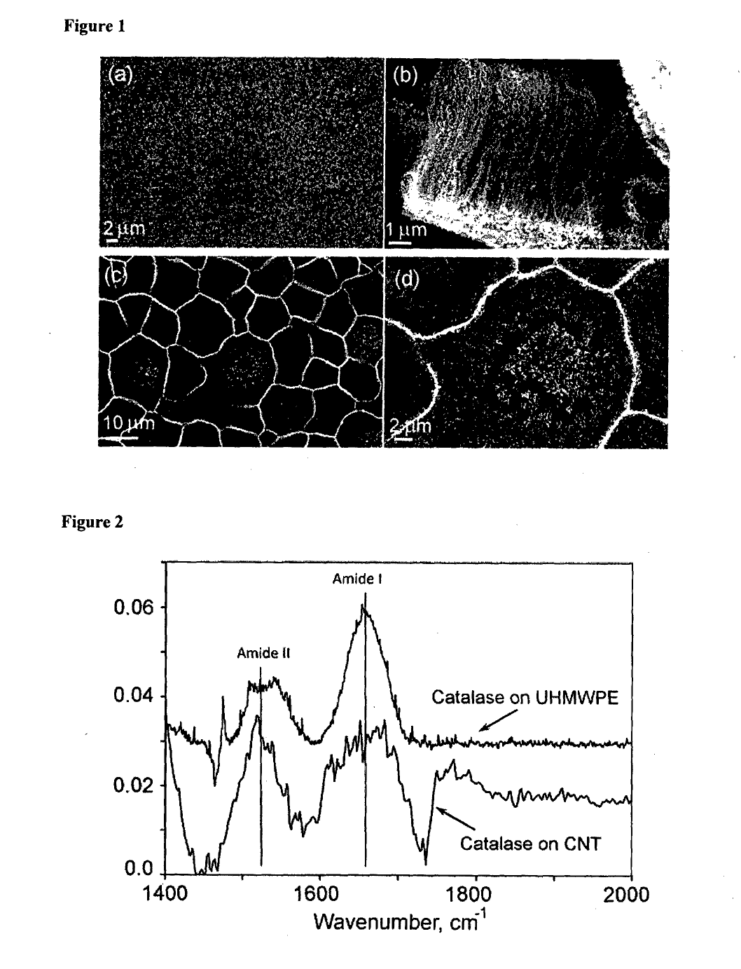

Initially, carbon nanotube arrays were grown on catalyst (iron particles) coated silicon substrates measuring 1×1 cm. The carbon nanotubes were grown using chemical vapour deposition in a thermal furnace in anacetylene+hydrogen atmosphere at a temperature 750° C. FIG. 1 shows top and side views of the as-prepared array. From these images one can see that the arrays exhibit a very high density of vertically-aligned nanotubes, yet somewhat twisted and entangled, with a mean length reaching 20 μm. In this example, the nanotubes have a small number of walls, typically about 5 (see FIG. 1), and a very low level of intrinsic defects.

The arrays were then incubated in a buffer solution of horseradish peroxidase (HRP) (50 ug/ml HRP concentration in PBS buffer, pH 5.5, incubated overnight, then washed 6 times in PBS, with last wash in mQ water) or in catalase (200 ug/ml catalase concentration in PBS buffer, pH 5.5, incubated overnight, then washed 6 times in PBS, with last wash in mQ water). The samples were allowed to dry overnight prior to analysis. After analysis, several samples were washed in sodium dodecyl sulphate (SDS), which is a detergent capable of disrupting physical interactions and fully unfolding proteins (SDS 2% water solution, 70° C., 1 hour). In the experiments with blocking agent, the samples were incubated for 3 h in 10% ethanol solution of (2,2,6,6-tetramethylpiperidin-1-yl) oxyl (TEMPO) free radical trapper. After washing in ethanol, the sample was incubated in HRP solution as described above. SEM images of the CNT arrays after incubation and washing in SDS are shown in FIG. 1. The arrays were slightly collapsed after drying, but most of the nanotubes preserved their vertical orientation.

FIG. 2 shows attenuated total reflection (ATR)—Fourier transform infrared (FTIR) spectra taken from the samples incubated in catalase solution. The lines at 1650 and 1540 cm⁻¹ are attributed to the Amide I and Amide II vibrations of amide bond in the protein back bone. A spectrum from Pill treated ultra-high-molecular-weight polyethylene (UHMWPE) with a monolayer of immobilized catalase is shown for comparison. Spectra taken from a silicon wafer and control CNT sample after incubation in buffer without protein were subtracted. The presence of the amide lines indicates that catalase is present.

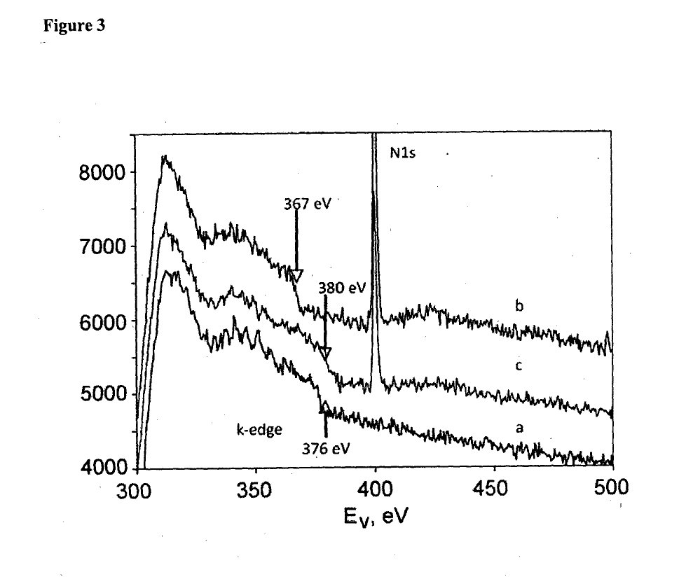

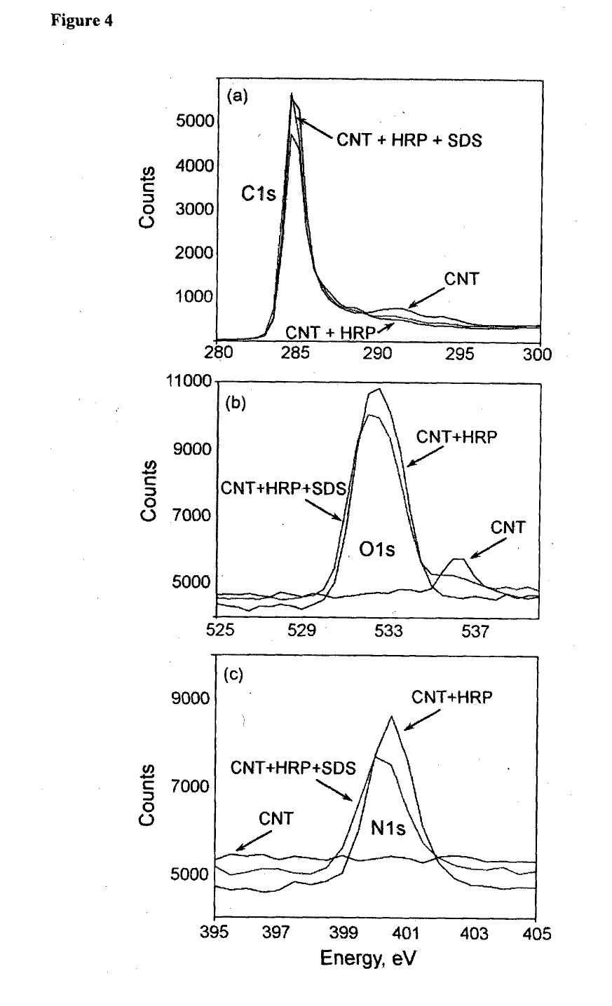

X-ray photoelectron spectroscopy (XPS) was used to study the chemical composition of the samples prior to and after incubation and detergent washing cycles (FIGS. 3 and 4). The strong C1s peak at 285 eV originates from the carbon nanotubes. The XPS spectrum of the CNT substrate shows an O1s oxygen peak attributed slight oxidation of the graphite-like carbon layer and does not show a nitrogen peak. The shoulders of the carbon C1s peak at 287 and 288 eV, corresponding to carbon in C—O and C═O groups of the protein molecules, appear only after incubation in HRP enzyme solution. The strong nitrogen peak at 400 eV and the strong oxygen peak at 532 eV that also appear after incubation in HRP solution are attributed to oxygen and nitrogen atoms in protein molecules. After washing of samples in SDS, these peaks remain in the spectra indicating that a fraction of the HRP enzyme is covalently immobilized.

FIG. 5 shows the nitrogen and oxygen compositions of the samples calculated from the XPS spectra. Very weak oxygen and nitrogen peaks can be noticed before protein attachment. After protein attachment, strong O1s and N1s signals appear. After washing with detergent, the intensity of the O1s and N1s peaks slightly decreases, but remains strong.

The concentration of oxygen in CNT is about twice of level of detection. The concentration of nitrogen is zero. After HRP attachment, the surface concentration of oxygen and nitrogen increases to 5.5% and 3.7%. After washing in SDS detergent, the surface concentration of oxygen decreases to 4.8% and the concentration of nitrogen decreases to 2.5%. The concentration of oxygen can be influenced by oxidation of the substrate during manipulations, while the concentration of nitrogen is associated only with amount of protein immobilized. Changes in the nitrogen concentration indicate that about 33% of the HRP protein is removed in the SDS detergent wash while 67% is strongly bonded to the material.

The inventors have predicted that the covalent bonding of proteins in this platform is based on free radical reaction. To prove this, free radicals in CNT samples were blocked with a free radical trapper agent. The intact CNT samples were treated by (2,2,6,6-tetramethylpiperidin-1-yl)oxyl and then incubated and washed; the amount of removed protein in the detergent wash increases to 88% with only 18% being strongly bonded on the samples. The inventors believe that the bonding mechanism is the same as that described in [4]. The residual bonding that occurs after application of the trapping agent is expected due to the continuous emergence of radicals. The plasma treatment of the unprotected carbon-covered surfaces creates radicals embedded in the surface which provide efficient covalent bonding of proteins. Specifically, on untreated surfaces 100% of the adsorbed proteins are washed away by the method described [4]. In contrast, plasma treated surfaces typically retained over 50% of the adsorbed protein layer indicating that these were covalently bonded [4]. In the platform reported here, about 67% of the adsorbed layer was covalently bonded and this dropped to 18% of the adsorbed layer when the surface was exposed to a radical trapping agent prior to incubation with the protein. This shows that the covalent bonding of proteins occurs due to a radical reaction resulting in efficient protein immobilization on this platform, as described in [4].

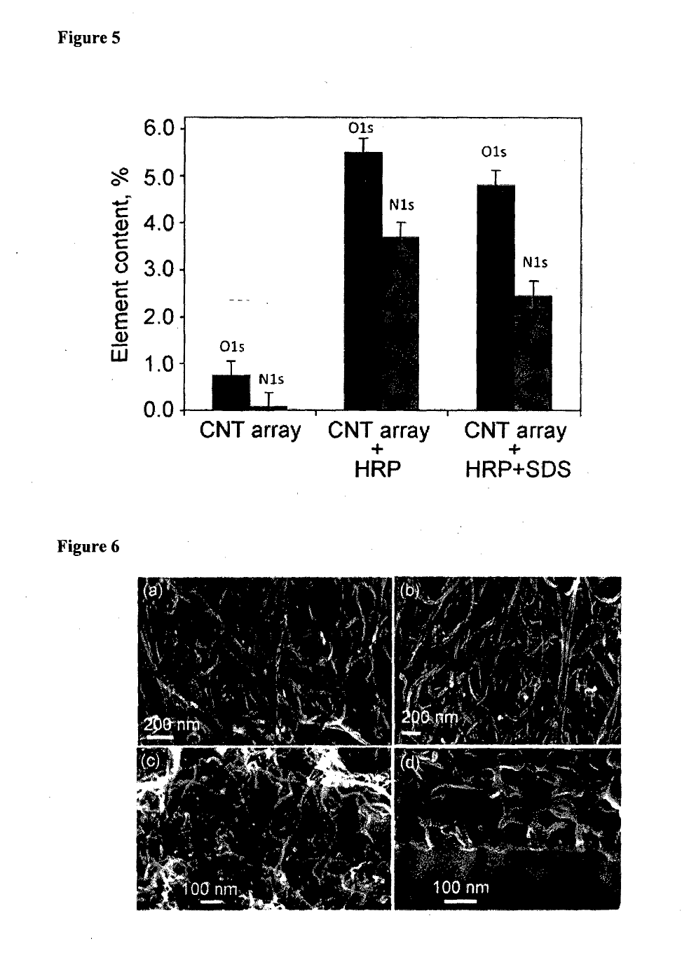

High-resolution SEM (HRSEM) images of the nanotubes with the attached catalase enzymes are shown in FIG. 6. The catalase molecules appear as small balls with a diameter of about 20 nm, corresponding to the size of catalase macromolecules.

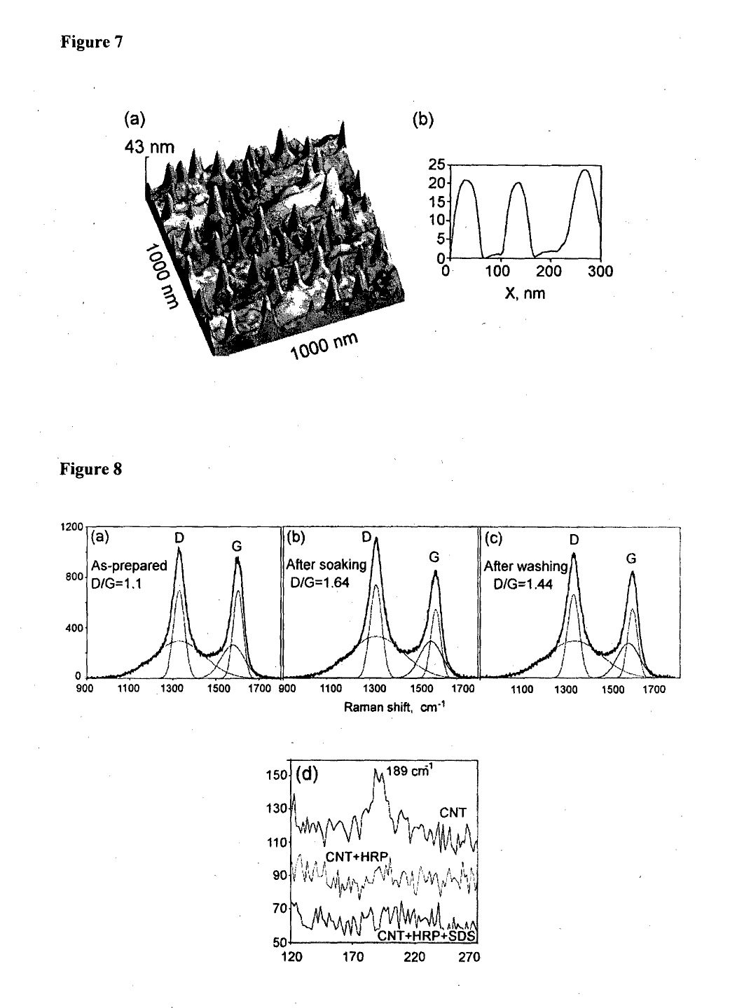

FIG. 7 shows an AFM image of catalase macromolecules adsorbed on a smooth polystyrene film. Their size agrees well with the size of the features observed in the high resolution SEM characterisation of the bottom of the CNT array samples (FIG. 6c, d).

To obtain an image of the bottom of the substrate, the nanotubes were removed mechanically. SEM images were taken from above looking down at the surface and from the edge of the cracked substrate. A large number of catalase molecules can be seen on the carbon coated surface of the substrate. The inventors have concluded from these images that most of the protein molecules are attached to the carbon film under the CNT array and not to the CNTs themselves.

Raman spectra of the initial CNT samples show D and G peaks attributed to the CNTs and the carbon film underneath (FIG. 8). The peaks have a mixture of broad and narrow components. For analysis, the Raman spectra were fitted with 4 Gaussian functions. The two narrow peaks at 1328 cm⁻¹ (D-peak) and 1602 cm⁻¹ (G-peak) are attributed to vibrational modes in the graphene structures in the CNT walls, and the two broad peaks at 1327 cm⁻¹ (D-peak) and 1577 cm⁻¹ (G-peak) are attributed to carbon in the film underneath. All of the samples give a strong G peak which is evidence of the high-quality structure of the carbon nanotubes. The influence of incubation in protein solution and subsequent SDS washing on the Raman spectra can be observed as a change in the DIG intensity ratio. This ratio is 1.1 for the virgin CNT samples and increases to 1.64 after immobilization of the protein molecules. It is then partially restored to 1.44 after washing the samples with SDS. This indicates that (i) proteins adsorbed on the nanotube surface disturb the vibrations of the CNTs; (ii) a considerable fraction of the protein adsorbed on the CNTs is removed from the samples by SDS washing (and hence, was attached non-covalently).

A signature of the radial breathing mode (RBM) was also found in the Raman spectra (FIG. 8). The inventors note here that the arrays comprise multi-walled nanotubes of 10 nm dia., thus consisting of a few layers. The RBM peak at 189 cm⁻¹ is quite strong for the as-grown nanotubes, indicating that the nanotube walls have only a few graphene layers. The RBM disappears after incubation in protein solution, indicating a disordering of the CNTs. The fact that the RBM is not recovered after SDS washing indicates that the CNTs remain disturbed.

Another characteristic of CNT ordering is π-plasmon shoulder of the XPS C1s line (FIG. 3). The π-plasmon is characteristic of electron excitation in regular graphitic structures like CNT's. As shown in FIG. 3, the regularity decreases when protein is attached, and the regularity recovers when the protein is washed off. In the spectra of virgin CNT samples, the π-plasmon shoulder extends to 376 eV. In spectra obtained after protein incubation, the π-plasmon shoulder is shorter, ending at 367 eV, and it is restored upon SDS washing to 380 eV.

Thus, the spectral investigation and SEM images have shown that the proteins are physically adsorbed on CNTs, and more strongly immobilized on the carbon film beneath the CNTs (via covalent attachment).

This example shows a strong attachment of horseradish peroxidase and catalase proteins on a graphite-like carbon film covered by densely packed, long multi-walled carbon nanotubes. Some protein molecules are initially physically adsorbed on the CNTs, as shown by the change in the D/G peak ratio, disappearance of the radial breathing mode in the Raman spectra, and the shortening of the rc-plasmon in XPS. However, most of the enzyme was removed from the CNTs by washing in SDS detergent, resulting in partial recovery of the Raman and XPS signals associated with regular CNT structure. The large (and relatively unchanged by SDS washing) protein signatures observed by FTIR ATR and XPS indicate that the majority of the enzyme molecules are covalently attached on the amorphous carbon film beneath the CNTs. It is believed that open graphitic edges rich with unpaired electrons (radicals), in the predominantly sp2 bonded film facilitate covalent enzyme immobilisation.

It is evident that the presently described structures will be useful as a support for the immobilisation of enzymes for continuous flow processing. The gaps between CNTs are about the same size as the diameter of the tubes 10-20 nm size. Accordingly, this should allow dissolved reactants to reach the carbon layer beneath the CNTs where most of the enzymes are immobilized whilst blocking bacteria whose size is typically in the 200-2000 nm range. The height of the CNTs is about 10 μm, much higher than the enzyme molecules (10-20 nm) so it is believed damaging microbes cannot approach the enzymes. It is expected that the tall densely packed CNT array or “forest” will also protect enzyme molecules against the hydrodynamic pressure caused by fluid flow, if the enzyme-rich substrate is used in continuous flow biochemical reactors.

Furthermore, the fact that the CNTs remain undisturbed and are believed to hide the attached proteins from microbial attack, illustrates utility in enzymatic processing applications. These same characteristics will also be valuable when the structures of the invention are utilised in other contexts, such as in biosensors, diagnostics, micro/nanofluidic or medical devices, where it is desirable for protection or framing of biological molecules to be provided.

### Example 2

### Mechanical Stability and Protective Capabilities of CNT Array Covered Substrates

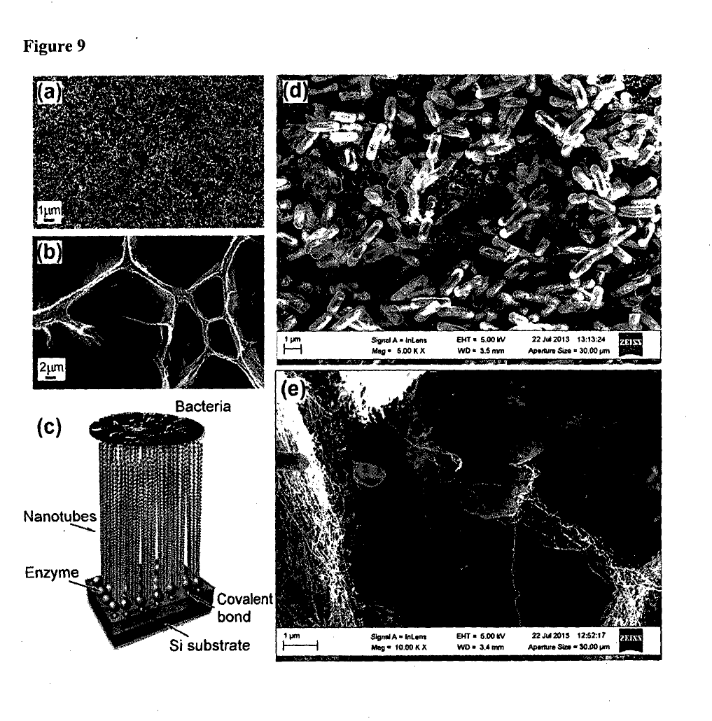

To investigate the mechanical stability of the hybrid platform, the enzyme-treated samples were placed in 24 well plates on a shaker (Benchtop Excella E1 shaker, New Brunswick Scientific) set to 30 rpm rotation with an amplitude of 2 cm. The plates were filled with LB solution (Peptone 10 g; yeast extract 5 g, and NaCl 10 g per litre of water), so the samples were completely immersed in the liquid. The samples were tested in this regime for 12 h. After that, to examine the sample structure, the samples were dried by the critical point drying (CPD) procedure (note that the carbon nanotube samples usually collapse during drying in open air). The samples were immersed in 100% ethanol, then transferred to the CPD chamber (BAL-TEC CPD030 Critical Point Dryer) and dried using liquid CO₂ for 3 h at the critical point (+31.1° C., 1000 PSI). After that, SEM was used to examine the structure of the CNT pattern. It was found that the pattern was not changed. A SEM image of the top surface of the CNT pattern after 12-h treatment in the shaker is shown in FIG. 9(a). The SEM image of the collapsed sample after drying in open air is shown in FIG. 9(b).

To check the protective capabilities of the platform a gainst microbiological attack, a direct experiment on the interaction of live bacteria (Bacillus subtilis, B. subtilis) was conducted with the platform. For this, samples with a live culture of B. subtilis were incubated for 12 h and then SEM images were produced after critical point drying (see the relevant description above). A schematic diagram of the immobilized enzyme protection against microbiological attack is shown in FIG. 9(c). This experiment has demonstrated that the bacteria cannot penetrate into the protective CNT forest, and are trapped on its surface, as seen in FIGS. 9(d) and (e).

### REFERENCES

1. G. Che, B. B. Lakshmi, C. R. Martin, and E. R. Fisher, Rodney S. Ruoff, Chemical Vapour Deposition Based Synthesis of Carbon Nanotubes and Nanofibers Using a Template Method, Chem. Mater. 1998, 10, 260-267.
2. K. B. K. Teo, C. Singh, M. Chhowalla, W. I. Milne, Catalytic Synthesis of Carbon Nanotubes and Nanofibers, in Encyclopaedia of Nanoscience and Nanotechnology, Ed. by H. S. Nalwa, vol. 10, 1-22 pp., 2003.
3. Jan Prasek, Jana Drbohlavova, Jana Chomoucka, JaromirHubalek, OndrejJasek, Vojtech Adam and Rene Kizek, Methods for carbon nanotubes synthesis—review, J. Mater. Chem., 2011, 21, 15872.
4. Bilek M M M, Bax D V, Kondyurin A, Yin Y, Nosworthy N J, Fisher K, et al. Free radical functionalization of surfaces to prevent adverse responses to biomedical devices. Proc Nat AcadSci USA 2011; 108:14405-10.

1. A structure comprising:
a substrate having a nanostructured spacer on a surface thereof; and
an organic molecule immobilised on the surface of the substrate.

2. The structure of claim 1, wherein the nanostructured spacer comprises a carbon nanotube array.

3. The structure of claim 2, wherein the carbon nanotubes of the carbon nanotube array are aligned substantially perpendicular to the surface of the substrate.

4. The structure of claim 1, wherein the organic molecule is covalently attached to the surface of the substrate.

5. The structure of claim 1, wherein the surface of the substrate comprises a carbon containing layer.

6. The structure of claim 5, wherein the organic molecule is covalently attached to the carbon containing layer.

7. The structure of claim 5, wherein the carbon containing layer includes any one or a combination of amorphous carbon, graphitic carbon, nano graphite or glassy carbon.

8. The structure of claim 1, wherein the organic molecule is a functional biological molecule.

9. The structure of claim 8, wherein the functional biological molecule is an enzyme.

10. The structure of claim 2, wherein average distance between the carbon nanotubes of the array is less than about 500 nm.

11. The structure of claim 2, wherein average distance between the carbon nanotubes of the array is from about 5 nm to about 100 nm.

12. The structure of claim 2, wherein average distance between the carbon nanotubes of the array is from about 10 nm to about 50 nm.

13. The structure of claim 2, wherein the carbon nanotubes have an average diameter of from about 1 nm to about 100 nm.

14. The structure of claim 2, wherein the carbon nanotubes have an average diameter of from about 10 nm to about 20 nm.

15. The structure of claim 2, wherein the carbon nanotubes have an average length of greater than about 500 nm.

16. The structure of claim 2, wherein the organic molecule is immobilised on the surface of the substrate after the carbon nanotubes are formed on the substrate.

17. The structure of claim 1, wherein the organic molecule is a catalytic molecule and the structure is for use in continuous flow processing.

18. The structure of claim 1, wherein the organic molecule is a functional biological molecule and the structure is for use in a diagnostic array or biosensor.

19-20. (canceled)

21. A method of protecting an organic molecule, the method comprising immobilising the organic molecule to a surface of a substrate, the substrate comprising a nanostructured spacer on the surface.

22. A method of protecting an organic molecule as in claim 21, wherein the nanostructured spacer is an array of carbon nanotubes.

23. A method as claimed in claim 21, wherein the organic molecule is a catalytic molecule and the method is for protecting the catalytic molecule during continuous flow processing.

24. The method of claim 23, wherein the catalytic molecule is an enzyme.

25. A method of producing a structure, the method comprising immobilising an organic molecule on a surface of a substrate, the surface of the substrate including a nanostructured spacer formed thereon.

26. A method as in claim 25, further comprising the step of forming the nanostructured spacer on the surface of the substrate.

27. A method as in claim 25, wherein the nanostructured spacer is an array of carbon nanotubes.

28. A method as claimed in claim 25, wherein the organic molecule is catalytic molecule.

29. A method as in claim 25, wherein the organic molecule is an enzyme.

30. A device comprising a structure of claim 1.

31. The device of claim 30 that comprises an enzyme and is for use in continuous flow processing.

32. The device of claim 30 which is a diagnostic kit or detection device, a tissue, cell or organ culture scaffold or support, a biosensor, an analytical plate, an assay component or a medical device.

33. The device of claim 32 which is a biosensor.
